(19) United States
(12) Patent Application Publication
(10) Pub. No.: US 2015/0352057 A1
(43) Pub. Date: Dec. 10, 2015

(54) METHOD OF USING PEELABLE FILM-FORMING COMPOSITION

(71) Applicant: JOHNSON & JOHNSON CONSUMER INC., Skillman, NJ (US)

(72) Inventors: Prashant Bahadur, Plainsboro, NJ (US); Jeffrey M. Wu, Princeton, NJ (US); Kamesh R. Yerramilli, Belle Mead, NJ (US)

(73) Assignee: JOHNSON & JOHNSON CONSUMER INC., Skillman, NJ (US)

(21) Appl. No.: 14/828,837

(22) Filed: Aug. 18, 2015

Related U.S. Application Data
(63) Continuation of application No. 14/264,029, filed on Apr. 28, 2014.
Application No. 14/264,029 is a continuation of application No. 13/631,144, filed on Sep. 28, 2012, now abandoned.

(51) Int. Cl. A61K 9/70; A61K 47/32

(52) U.S. Cl. CPC A61K 9/7015; A61K 47/32

(57) ABSTRACT

The compositions and methods of this invention relate to peelable film-forming compositions that may be applied to and removed from the skin containing polyvinyl alcohols and adhesion-enhancing polymers.

### FIELD OF THE INVENTION

The method of this invention uses peelable film-forming compositions that can be applied to skin for treating and removing comedones.

### BACKGROUND OF THE INVENTION

Open comedones, or blackheads, are a primary sign of acne vulgaris, a disease of the hair follicles of the face, chest and back that affects a vast majority of humans during their lifetimes. A blackhead consists of a widened hair follicle filled with skin debris, bacteria and sebum and is capped with a blackened mass of skin debris. Blackheads can be unsightly and cause embarrassment to the individual who suffers with this condition.

In the past, blackhead sufferers have used skin cleansing products, including astringents, toners, surfactant-containing cleansers and the like, to cleanse and treat their skin daily. They have also utilized exfoliating implements and compositions containing exfoliating ingredients to remove dead skin and improve the tone and texture of their skin.

Blackhead treatment has been challenging in the past due to a poor understanding of blackhead formation physiology. One example of an implement used to treat and prevent blackhead formation is set forth in U.S. Pat. No. 5,139,711. This patent describes a face mask that contains hydrolyzed grain end products, seaweed derivatives and water. The mask is intended to be applied to the skin, permitted to dry and then rinsed off with water, assisting in the removal of dead surface skin cells and other debris.

Other methods for exfoliation are set forth, for example, in U.S. Patent Publication No. 2004/0057921. This publication describes scrub formulations that contain polyethylene beads to help reduce the occurrence of blackheads.

Yet another method for treatment of blackheads is set forth in U.S. Pat. No. 5,512,277, which describes a keratolytic plug remover composition containing a polymer with a salt forming group. This composition forms a film which can be applied to the skin, dried and then peeled off the skin.

However, it would be desirable to have a product that provides both the mechanical action to remove blackheads and to provide skin exfoliation. The mechanical action would provide immediate or fast removal of blackheads, while chemical exfoliation using an active ingredient such as salicylic acid would provide a continuous action to remove excessive dead skin cells to prevent further blackhead formation. Such a combination of mechanisms of action is not known heretofore.

### SUMMARY OF THE INVENTION

The compositions and methods of this invention relate to peelable, adhesive film-forming compositions comprising, consisting essentially of and consisting of:

(a) An anti-acne agent selected from the group consisting of: salicylic acid, sulfur and one or more alpha hydroxyacids;

(b) A high molecular weight polyvinyl alcohol component, which may be partially hydrolyzed, having a molecular weight from about 80,000 to about 200,000 in the amount of from about 10 to about 18% by weight of the composition;

(c) An adhesion enhancing polymer selected from the group consisting of polyesters, polyacrylates, polyvinylacrylates, polyurethanes, polyvinylcaprolactam and a combination thereof, said adhesion enhancing polymer component being present in the composition in the amount of from about 0.5 to about 4% by weight of the composition; and

(d) A low molecular weight partially hydrolyzed polyvinylalcohol component, which may be partially hydrolyzed as described herein, having a molecular weight from about 10,000 to about 50,000 in an amount of from about 0 to about 5% by weight of the composition; wherein the total weight percent of components (b) and (c) in the composition does not exceed 20%; wherein the total weight percent of components (b) and (d) in the composition does not exceed 20%; and wherein the total weight percent of components (b), (c) and (d) does not exceed 25% of the composition. Preferably, the high and low molecular weight polyvinyl alcohols useful in the compositions of this invention are hydrolyzed to a degree of from about 50% to about 98%, more preferably from about 75% to about 95% and most preferably, from about 87% to about 89%.

### DETAILED DESCRIPTION OF THE INVENTION

The compositions used in this invention preferably contain at least one high molecular weight polyvinyl alcohol, having a structure as set forth below:

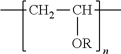

wherein R is hydrogen or —COCH₃, and

wherein n is from about 900 to about 4500 when R is hydrogen, and from about 20 to about 1110 when R is COCH₃. The polyvinyl alcohols useful in the compositions and methods of this invention may be partially or completely hydrolyzed.

Preferably, the compositions used in this invention comprise, consist essentially of and consist of at least one polyvinyl alcohol having a molecular weight from about 80,000 to about 200,000 and, preferably, a degree of hydrolysis from about 50% to about 98%, more preferably from about 75% to about 95% and most preferably 87% to about 89%, in the amount of from about 10 to about 18% by weight of the composition. Preferably, the high molecular weight polyvinyl alcohols are present in the compositions of this invention in an amount of from about 12 to about 16% by weight of the composition. More preferably, they are present in an amount of from about 13 to about 15% by weight of the composition.

Optionally, the compositions used in this invention comprise, consist essentially of and consist of a low molecular weight polyvinyl alcohol of the following structure:

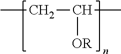

wherein R is hydrogen or —COCH₃, and

wherein n is from about 170 to about 1100 when R is hydrogen and from about 2 to 300 when R is COCH₃. Such low molecular weight polyvinyl alcohols may be partially or completely hydrolyzed.

Preferably, the compositions used in this invention comprise, consist essentially of and consist of at least one polyvinyl alcohol having a molecular weight from about 10,000 to about 50,000 and, preferably, a degree of hydrolysis from about 50% to about 98%, more preferably from about 75% to about 95% and most preferably 87% to about 89% in the amount of from about 0 to about 5% by weight of the composition. Although a second, lower molecular weight polyvinyl alcohol (i.e., about 10,000 to about 50,000) may be present in the compositions of this invention, it is not necessary to be present in order to achieve the superior results of the compositions of this invention.

If a low molecular weight polyvinyl alcohol is present in the compositions used in this invention, the ratio between the high and low molecular weight polyvinyl alcohols should be from about 2:1 to about 3:1.

The compositions used in this invention preferably further comprise, consist essentially of and consist of an adhesion enhancing polymer selected from the group consisting of polyesters, polyacrylates, polyvinylacrylates, polyurethanes, polyvinylcaprolactam and a combination thereof.

The compositions used in this invention preferably contain at least one polyvinylcaprolactam, having a structure as set forth below:

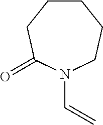

Preferably, the compositions used in this invention comprise, consist essentially of and consist of at least one polyvinylcaprolactam, having a molecular weight about 150 g/mol in the amount of from about 0.5 to about 4% by weight of the composition.

The adhesion enhancing polymer component should preferably be present in the composition in the amount of from about 0.5 to about 4% by weight of the composition. More preferably, it should be present in the composition in the amount of from about 0.5 to about 2% by weight of the composition.

As used herein, the term “adhesive force” refers to the force required to remove from the skin a material that has been adhered, or adhesively fastened, to the skin.

As used herein, the term “film” refers to a composition that forms a thin layer or membrane on mammalian and, more particularly, human skin.

As used herein, the term “peelable” refers to materials that can be removably applied to and adhere to surfaces such as the surface of mammalian and human skin and can be subsequently removed from the skin by force. Such peelable materials can be effective to exfoliate skin and remove comedones by mechanical means. Peelable films according to the compositions and methods of this invention can be adhesively and removably applied to human skin and, by virtue of being forcibly removed, carry away the dead skin cells, hair, dirt, sebum and blackheads which have adhered to the films while leaving behind the skin benefit agents and actives onto the skin. In order to achieve this activity, the peelable films of this invention should have an adhesive force of at least about 5 to about 25 ounces (“oz.”). The peelable films of this invention should not have such high adhesive force that they cause disruption to the outer layer of the skin and thereby damage the skin. Likewise, the adhesive force of the peelable films of this invention should be sufficiently high to enable the films to exfoliate and remove comedones from the skin.

The compositions used in this invention preferably contain a film-forming polymer that functions to enhance film formation and provide some water resistance. “Film-forming polymer,” as used herein, refers to polymers that when dissolved in the composition, permit a continuous or semi-continuous film to be formed when the composition is spread onto a surface such as a skin surface and the liquid vehicle is allowed to evaporate. As such, the polymer should dry on the skin surface in need of treatment such that it is spread in a predominantly continuous manner, rather than in such a way that the composition forms a plurality of discrete, island-like structures. Generally, the films formed by applying compositions on the skin according to embodiments of the invention described herein, are less than, on average, about 100 microns in thickness, and preferably less than about 50 microns.

The film-forming agent should preferably be present in the composition in the amount of from about 1 to 10% by weight of the composition.

The compositions used in this invention optionally further comprise a solvent/diluents and delivery vehicle. Upon evaporation and concentration of the solvent, the compositions used in this invention can form a film that adheres to the skin. Film-forming polymers provide an additional benefit that, upon exposure to the skin and the air, the compositions used in this invention form a film that can be subsequently removed, leaving behind the actives on the skin and/or simultaneously removing undesirable materials from the skin. The film can be easily removed with water or can be peeled off.

Polyvinyl alcohol compounds as set forth above act as preferred film-forming polymers in the compositions of this invention.

The compositions used in this invention further comprise a skin benefit agent. A “skin benefit agent” as used herein refers to a cosmetic formulation and is not considered to be directed to the treatment of a particular condition, but possesses multifunctional properties that can contribute to the improvement of a condition. For example, glycerol (glycerin) can be included in a moisturizing cream (having a specific moisturizing agent) as an excipient, and in addition the glycerol contributes to and enhances the moisturizing properties of the cream.

One such a benefit agent is a skin conditioning agent. A “conditioning agent” as used herein refers to substance that improves the quality of the skin surface, corrects or prevents skin damage. In particular, conditioning agent maintains the moisture of the skin when the film is peeled.

Examples of conditioning agents include cationic compounds with quaternary ammonium functional groups such as Polyquaternium-39 and the like.

The skin conditioning component should preferably be present in the composition in the amount of from about 1 to 10% by weight of the composition, preferably from about 2 to about 8% by weight of the composition and more preferably from about 3 to about 6% by weight of the composition.

The compositions used in this invention comprise an active agent. An “active ingredient” or agent used herein refers to a substance that presents specific properties used for the treatment of a particular condition. These active agents can be pharmaceutical agents, cosmetics, or wound healing agents.

Active ingredients to be delivered through the peelable film according to the invention can be any of a pharmaceutical or a cosmetic agent that are compatible with the film-forming polymers and adhesion enhancing polymers. Examples of active agent can be an anti-inflammatory, an antioxidant, antimicrobial a moisturizing agent, an anti-hyperpigmentation agent, an anti-blotching agent, an anti-aging agent, an anti-collagenase substance, a free radical scavenger, a seboregulator, an hydrative, a keratolytic agent and an α- or β-hydroxy acid and the like.

Anti-acne/keratolytic agents include salicylic acid, benzoyl peroxide, sulfur, retinoic acid, alpha hydroxyacids and any of several fruit acids and the like.

The compositions used in this invention optionally further comprise a color enhancing agent such as coloring agent, opacifier, and pearlizer. Such color enhancing agent include, but not limited to, zinc striate, magnesium stearate, titanium dioxide, EGMS (ethylene glycol monostearate) or Lytron 621 (Styrene/Acrylate copolymer); all of which are useful in enhancing the appearance or cosmetic properties of the skin peelable composition.

The methods of this invention include the use of the compositions of this invention to remove undesirable materials from the skin or other surface and/or to deposit benefit agents to the surface. Preferably, the individual wishing to utilize the compositions and methods of this invention first wash his or her face with a regular, non-acne cleanser And then gently wipe with a towel until fully dry. A marble sized amount of composition according to this invention is squeezed onto the finger tip and gently applied to the desired skin surface to form a smooth, even layer. The composition is permitted to dry for 10 to 15 minutes. Once the composition is dry, the peel is gently rolled from the edge of the dried film and peeled off the skin in an upward motion.

The following are illustrative examples of the compositions of this invention and the methods of using such compositions. They are intended to be merely illustrative, and not limitative of the remainder of the disclosure in any way whatsoever.

### EXAMPLE 1

An adhesive, peelable film-forming composition according to this invention was made by combining the ingredients set forth in Table 1 below at a temperature of 80-95° C., under pressure ambient pressure according to the following method.

For Phase A, Water was added to the beaker with propeller mixing. Subsequently, Glycerin was added to the beaker and the mixture blended until uniform. After the mixture achieved uniformity, Disodium EDTA was added and permitted to dissolve. Then, propylene glycol and olefin sulfonate-coated TiO₂ were added and the mixture blended until uniform. Once the batch was uniform, a higher molecular weight partially hydrolyzed polyvinyl alcohol was added and dispersed uniformly. Once the batch was uniformly dispersed, a lower molecular weight partially hydrolyzed polyvinyl alcohol was added to the mixture and dispersed uniformly. The batch was then heated to 85° C. (the minimum cooking temperature of the partially hydrolyzed polyvinyl alcohols) and held at that temperature for 45 minutes. After 45 minutes of heating at 85° C., Polyquaternium-39, a terpolymer of acrylic acid, acrylamide and diallyldimethylammonium chloride was added to the beaker and the batch mixed until uniform. Once uniform, the mixture is cooled to below 35° C. and the batch held at that temperature for phasing with Phase B.

Phase B was made by adding to a separate beaker ethyl alcohol, polyvinylcaprolactum, salicylic acid and fragrance. This blend was mixed until uniformly dissolved.

Very slowly, Phase B was added to Phase A and the resulting batch mixed until it formed a homogeneous gel.

The following compositions were made in accordance with the above-described method using the ingredients set forth in Table 1 below.

TABLE 1
Formulations containing two partially hydrolyzed
Polyvinyl alcohols (high molecular weight and low
molecular weight) with Polyvinylcaprolactam (Example B)
and without Polyvinylcaprolactam (Example A)

| Chemical Names | Formula A | Formula B |
|---|---|---|
| Water | 45.150 | 44.150 |
| Glycerin | 4.000 | 4.000 |
| Partially hydrolyzed Polyvinyl | 13.500 | 13.500 |
| Alcohol (Higher Molecular Weight) | | |
| Partially hydrolyzed Polyvinyl | 5.000 | 5.000 |
| Alcohol (Lower Molecular Weight) | | |
| Polyquaternium-39 | 1.500 | 1.500 |
| Ethyl Alcohol | 30.000 | 30.000 |
| Polyvinylcaprolactam | 0.000 | 1.000 |
| Salicylic Acid | 0.500 | 0.500 |
| TiO2, Sodium C14-16 Olefin | 0.200 | 0.200 |
| Sulfonate | | |
| Disodium EDTA | 0.050 | 0.050 |
| Fragrance | 0.100 | 0.100 |
| Total % | 100.000 | 100.000 |

TABLE 2
Formulations without the lower molecular weight
partially hydrolyzed Polyvinyl alcohol with
Polyvinylcaprolactam (Example D) and without
(Example C) Polyvinylcaprolactam

| Chemical Names | Formula C | Formula D |
|---|---|---|
| Water | 48.75 | 47.75 |
| Glycerin | 4.000 | 4.000 |
| Propylene Glycol | 1.0 | 1.0 |
| Magnesium Aluminum Silicate | 0.4 | 0.4 |
| Partially hydrolyzed Polyvinyl | 13.500 | 13.500 |
| Alcohol (Higher Molecular Weight) | | |
| Partially hydrolyzed Polyvinyl | 0.000 | 0.000 |
| Alcohol (Lower Molecular Weight) | | |
| Polyquaternium-39 | 1.500 | 1.500 |
| Ethyl Alcohol | 30.000 | 30.000 |
| Polyvinylcaprolactam | 0.000 | 1.000 |
| Salicylic Acid | 0.500 | 0.500 |
| TiO2, Sodium C14-16 Olefin | 0.200 | 0.200 |
| Sulfonate | | |
| Disodium EDTA | 0.050 | 0.050 |
| Fragrance | 0.100 | 0.100 |
| Total % | 100.000 | 100.000 |

### EXAMPLE 2

### Adhesion Measurement

A human consumer test was conducted on five healthy male and two female subjects to determine the skin adhesion of polymer films formed by a composition of blackhead removing gel Formula B vs. formula A, prepared as in Table 1. Formula A did not contain Polyvinylcaprolactam (e.g. Luviskol-plus, commercially available from BASF Corporation). Formula B contained 1% Polyvinylcaprolactam. These formulas were uniformly applied to a preselected testing site with 0.6 grams on a circle of two-inch diameter on the forearm of the subjects. After the polymer film became completely dry, a handheld scale or weight meter (30 lbs Berkley) was applied to determine the force or required to remove the film from the skin vertically. This instrument measured the adhesion force of the polymeric film attaching to the skin. Skin adhesion for two similar Formulae (Formula C and Formula D) that did not contain a low molecular weight polyvinyl alcohol was also measured. Formula C did not have polyvinylcaprolactam while Formula D had 1% polyvinylcaprolactam.

As can be seen from Table 3, addition of polyvinylcaprolactam enhanced the skin adhesion for the formula B and Formula D as compared with the corresponding control compositions, i.e., Formula A and Formula C respectively.

TABLE 3
Adhesive Force Measurement

| | Adhesive | |
|---|---|---|
| | Force (oz) | |
| | Average based | |
| Formula | on 7 subjects | Comments |
| Formula | 3.5 +/− 1 | Formula with Two PVOH's and |
| A | | No Polyvinylcaprolactum |
| Formula | 10.5 +/− 1.5 | Formula with two PVOH + 1% |
| B | | Polyvinylcaprolactam |
| Formula | 1.5 +/− 1 | Formula with one PVOH and |
| C | | No Polyvinylcaprolactum |
| Formula | 8 +/− 0.5 | Formula with one PVOH + 1% |
| D | | Polyvinylcaprolactam |

### EXAMPLE 3

### Unexpected Effects of Blending Polyvinyl Alcohol (High Molecular Weight) and Polyvinylcaprolactam on the Adhesion Strength

Ten compositions, set forth in Tables 4 and 5,were made in accordance with the method set forth in Example 1. These compositions contained varying levels of high molecular weight Polyvinyl Alcohol and Polyvinylcaprolactam in the proportions set forth in the Tables 4 and 5 below and were uniformly applied to the forearm on a fixed area (0.6 grams on an area equal to the area of a circle of 2 inch diameter) on seven subjects. The polyvinyl alcohol used herein was partially hydrolyzed to the degree between 87% and 89%. After the polymer film dried completely, a handheld scale or weight meter (30 LBs Berkley) was used to determine the normal force required to remove the film from the skin. This force measures the adhesion force of the polymeric film attaching to the skin. From the results tabulated below, we observe that:

TABLE 4
Formulations made for studying the effect of blending
polyvinyl alcohol (higher molecular weight) and
Polyvinylcaprolactam on the adhesion strength

| Chemical Names | Formula |
|---|---|
| Water | 48.75 |
| Glycerin | 4.000 |
| Propylene Glycol | 1.0 |
| Magnesium Aluminum Silicate | 0.4 |
| Partially hydrolyzed Polyvinyl | See table 5 |
| Alcohol (Higher Molecular Weight) | |
| Polyquaternium-39 | 1.500 |
| Ethyl Alcohol | 30.000 |
| Polyvinylcaprolactam | See table 5 |
| Salicylic Acid | 0.500 |
| TiO2, Sodium C14-16 Olefin | 0.200 |
| Sulfonate | |
| Disodium EDTA | 0.050 |
| Fragrance | 0.100 |
| Total % | 100.000 |

TABLE 5
Amount of Polyvinyl Alcohol (Higher
Molecular Weight) and Polyvinylcaprolactam.

| | % Partially hydrolyzed | |
|---|---|---|
| | Polyvinyl Alcohol (Higher | % Polyvinyl |
| Formula | Molecular weight) | Caprolactam |
| 1. | 13.5 | 0 |
| 2. | 13.5 | 1 |
| 3. | 13.5 | 2 |
| 4. | 3 | 0 |
| 5. | 10 | 0 |
| 6. | 10 | 1 |
| 7. | 10 | 2 |
| 8. | 5 | 5 |
| 9. | 2 | 10 |
| 10. | 0 | 20 |

Adhesion strength of the Formulae set forth in Tables 4 and 5 was tested as set forth above. Results of these tests are recorded in Table 6 below.
a) By comparing Formulae 1, 4 and 5 (in table 6), it can be seen that in compositions that did not contain polyvinylcaprolactam, higher levels of Polyvinyl alcohol resulted in higher adhesion strength.
b) By comparing Formulae 1, 2 and 3, all of which contained 13.5% partially hydrolyzed polyvinyl alcohol, it can be observed that the adhesion strength increased in proportion to the increase in the amount of polyvinylcaprolactam present in the compositions.
   This clearly indicates that the combination of partially hydrolyzed polyvinyl alcohol and polyvinylcaprolactam in accordance with the compositions of this invention provide excellent adhesion to the skin. A similar trend can be seen with respect to Formulas 5, 6 and 7 which contain 10% partially hydrolyzed polyvinyl alcohol.

TABLE 6
Adhesion Strength

| | % Partially | | |
|---|---|---|---|
| | hydrolyzed Polyvinyl | | Adhesion |
| | Alcohol (Higher | % Polyvinyl | Strength |
| Formula | Molecular weight) | Caprolactam | (oz) (n =7) |
| 1. | 13.5 | 0 | 1.5 +/− 0.5 |
| 2. | 13.5 | 1 | 4.5 +/− 1.0 |
| 3. | 13.5 | 2 | 8.0 oz +/− 1.0 |
| 4. | 3 | 0 | 0 +/− 0.5 |
| 5. | 10 | 0 | 0.5 +/− 0.5 |
| 6. | 10 | 1 | 3 +/− 1 |
| 7. | 10 | 2 | 6.5 +/− 1 |
| 8. | 5 | 5 | 1.5 +/− 0.5 |
| 9. | 2 | 10 | 0.5 +/− 0.5 |
| 10. | 0 | 20 | 0.5 +/− 0.5 |

1. A method of treating skin with a peelable, adhesive film-forming composition, said method comprising:
spreading a composition comprising the following ingredients onto the skin:
(a) a high molecular weight polyvinyl alcohol;
(b) an adhesion enhancing polymer selected from the group consisting of polyesters, polyurethanes, polyvinylcaprolactam, and a combination thereof, said adhesion enhancing polymer component; and
(c) a low molecular weight polyvinylalcohol component;
allowing said composition to dry completely when applied to human skin to form a film; on the skin to form a film; and
peeling the film from the skin with an adhesive force of about 5 to about 25 ounces to remove the film with undesirable material from the skin without damaging the skin or disrupting the outer layer of the skin.

2-30. (canceled)

31. A peelable, adhesive film-forming composition comprising:
(b) n anti-acne agent selected from the group consisting of: salicylic acid, sulfur and one or more alpha hydroxyacids;
(c) A high molecular weight polyvinyl alcohol component having the structure 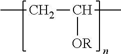
wherein R is hydrogen or —COCH₃, and
wherein n is from about 900 to about 4450 for polyvinyl alcohol R═H, and about 170 to about 1110 when R is COCH₃, said high molecular weight polyvinyl alcohol having a molecular weight from about 80,000 to about 200,000 in the amount of from about 10 to about 16% by weight of the composition;
(c) an adhesion enhancing polymer selected from the group consisting of polyesters, polyacrylates, polyvinylacrylates, polyurethanes, polyvinylcaprolactam and a combination thereof, said adhesion enhancing polymer component being present in the composition in the amount of from about 0.5 to about 4% by weight of the composition; and
(d) a low molecular weight polyvinylalcohol component having the structure 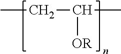
wherein R is hydrogen or —COCH₃, and
wherein n is from about 170 to about 1200 when R is hydrogen and from about 2 to 290 when R is COCH₃, said low molecular weight polyvinyl alcohol having a molecular weight from about 10,000 to about 50,000 in an amount of from about 0 to about 5% by weight of the composition;
wherein the total weight percent of components (b) and (c) in the composition does not exceed 20%; wherein the total weight percent of components (b) and (d) in the composition does not exceed 20%; and wherein the total weight percent of components (b) plus (c) plus (d) does not exceed 25%.

32. A composition according to claim 31 wherein the ratio of component (b) to component (d) in the composition is from about 1:1 to 6:1.

33. A composition according to claim 32 wherein the ratio of component (b) to component (d) in the composition is from about 2:1 to about 3:1.

34. A composition according to claim 31 wherein the anti-acne agent is salicylic acid, wherein said salicylic acid is present in the composition in an amount of from about 0.5% to about 2% by weight of the composition.

35. A composition according to claim 31 wherein the anti-acne agent is sulfur, wherein said sulfur is present in the composition in an amount of from about 1% to about 10% by weight of the composition.

36. A composition according to claim 31 wherein the anti-acne agent is one or more alpha-hydroxy acids, wherein said alpha-hydroxy acids are present in the composition in the amount of from about 5% to about 10% by weight of the composition.

37. A composition according to claim 31 wherein said adhesion enhancing polymer is polyvinylcaprolactam.

38. A composition according to claim 31 wherein the adhesion strength of the composition is at least 3.

39. A composition according to claim 31 wherein the high molecular weight polyvinyl alcohol has a degree of hydrolysis from about 50% to about 99%.

40. A composition according to claim 39 wherein said degree of hydrolysis is from about 75% to about 95%.

41. A composition according to claim 40 wherein said degree of hydrolysis is from about 75% to about 95%.

42. A composition according to claim 39 wherein said degree of hydrolysis is from about 87% to about 89%.

43. A composition according to claim 40 wherein said degree of hydrolysis is from about 87% to about 89%.
